(19) United States
(12) Patent Application Publication
(10) Pub. No.: US 2024/0299446 A1
(43) Pub. Date: Sep. 12, 2024

(54) COMPOSITION FOR PREVENTING, TREATING OR IMPROVING CALCIUM RELATED DEGENERATIVE DISEASES, OSTEOPOROSIS OR CANCERS COMPRISING SIGMA ANTI-BONDING MOLECULE CALCIUM GLUCONATE

(71) Applicant: Kyu Heon LEE, Coquitlam (CA)

(72) Inventor: Kyu Heon LEE, Coquitlam (CA)

(21) Appl. No.: 18/314,432

(22) Filed: May 9, 2023

(30) Foreign Application Priority Data
Mar. 6, 2023 (KR) 10-2023-0029170

(51) Int. Cl. A61K 33/10; A61K 35/614; A61K 35/618; A61K 47/26; A61P 19/10; A61P 35/00

(52) U.S. Cl. CPC A61K 33/10; A61K 35/614; A61K 35/618; A61K 47/26; A61P 19/10; A61P 35/00

(57) ABSTRACT

The present invention relates to a composition for preventing, treating or improving calcium-related degenerative diseases, osteoporosis or cancers, which includes Sigma Anti-bonding Calcium (SAC) gluconate as an active ingredient, and more particularly, to a composition for preventing, treating or improving calcium-related degenerative disease such as osteoporosis, as well as osteoporosis, generation of neoplasm such as malignant tumors, cancers, or the like, that provides calcium ions without side effects on cells through a calcium channel and glucose transport thus to normalize cellular metabolism and prevent degeneration thereof.

### CROSS REFERENCE TO RELATED APPLICATIONS

This application claims the priority benefit of Korean Patent Application No. 10-2023-0029170 filed on Mar. 6, 2023, the entirety of which is incorporated by reference herein.

### BACKGROUND OF THE INVENTION

### 1. Field of the Invention

The present invention relates to a composition for preventing, treating or improving calcium-related degenerative diseases, osteoporosis or cancers, which includes Sigma Anti-bonding Molecule Calcium gluconate as an active ingredient, and more particularly, to a composition for prevention, treatment or improvement of degenerative diseases such as osteoporosis, as well as osteoporosis, generation of neoplasm such as malignant tumors, cancer, etc. that provides calcium ions without side effects to cells through a calcium channel and glucose transport thus to normalize cellular metabolism and prevent degeneration thereof.

### 2. Description of the Related Art

Until now, in order to overcome cancers, numerous anticancer drugs and therapies including the first generation cytotoxic anti-cancer drugs, radiation therapy, targeted anticancer drugs, recent immune-oncology drugs, and even heavy ion therapy (“syncrotron”) have been developed. However, all of the therapeutics and therapies are still limited and have made patients suffer due to different adverse effects. Further, although a variety of medicines from the first generation of osteoporosis therapeutics to recent Denosumab RANKL inhibitor (receptor activator of nuclear factor kappa-B ligand) have been developed, these show only insignificant increase in bone mineral density even with involving adverse effects.

Osteoporosis, which is one of degenerative diseases, is a disease in relation to calcium metabolism, wherein the calcium metabolism is important for cellular metabolism. That is, osteoporosis is an incidental or accompanying disease due to lower calcium metabolism in cells rather than a disease of the bone itself. In fact, all diseases begin within the cell. Therefore, due to a calcium homeostasis problem in the cell, it causes a deterioration in function of mitochondria, variation in DNA replication, and some diseases including cancer, during cellular metabolism.

In other words, neoplasm cancers such as carcinoma, sarcoma, etc. are known as neoplasm pathologically caused by gene mutation, and such gene mutation is due to calcium homeostasis break down wherein calcium is known as a neurotransmitter. That is, when calcium homeostasis is broken down, gene mutation may occur. This means that gene replication would not be normally done when a calcium signal is instable during gene replication.

Rapid imbalance in calcium, which is typically shown in cancer patients, may additionally derive osteoporosis or induce calcification of cells due to an increase in the blood calcium content. Intracellular calcium influx is performed through both of calcium channels (legend calcium channel, and voltage calcium channel) present in a cell membrane, DNA of a tumor cell is repeatedly replicated using calcium signals and thus results in sharp cell proliferation. In this regard, the tumor cell secretes a parathormone (PHTr)-like material and further activates osteoclast cells to derive bone resorption (“osteolysis”) from the bone, which in turn allows the tumor cell to be provided with plenty of calcium (Endocrine Reviews, Volume 19, Issue 1, 1 Feb. 1998, Pages 1854).

Furthermore, osteoporosis occurs to increase the risk of bone fracture (Riggs BL. Osteoporosis. In: Textbook of Medicine (Wyngaarden JB, ed), 19th ed, Saunders, Philadelphia, 1992; pp 1426-1430).

Accordingly, it is now increasingly necessary to develop an improved therapeutic preparation for treatment of degenerative diseases such as osteoporosis without side effects, and pharmaceutical aids thereof.

On the ground of the requirement as described above, there is an invention in relation to an activated ionic calcium composition for preventing and treating osteoporosis, which was developed by the present inventor of the present disclosure (Korean Patent Laid-Open Publication No. 10-2014-0064262). However, this composition supplies calcium only through a calcium channel and still entails a disadvantage of being less effective in prevention, treatment or improvement of degenerative diseases such as osteoporosis, cancers, etc.

Further, according to the data introduced in the experiments and methods described in the above patent document, the basic dose is 0.0012%. This means that Sigma Anti-bonding Molecule Calcium Carbonate (SAC) was administered as a diluted solution, which was prepared by diluting the SAC crude solution at 1200 ppm in water by 100 times. That is, for adult dosing, 5 ml of crude solution (SAC 5 to 6 mg/5 ml) should be diluted 100 times with 500 ml of water, hence causing problems in that it is very inconvenient for individuals who cannot drink water too much, terminal cancer patients, renal failure patients, etc., and it is of little practical use.

Therefore, in order to investigate applicability of Sigma Anti-bonding Molecule Calcium Carbonate formed by combining SAC in D-glucose or L-glucose as a preparation for preventing, treating or improving calcium-related degenerative disease such as osteoporosis, as well as osteoporosis, cancers, etc., the present inventor has conducted experiments for not only biochemical markers in relation to bone turnover but also influence on BMD, bone and mineral metabolism by injecting the above preparation into the oral cavity of animals with osteoporosis, etc., and transferring the same into the cells through animal experiments, and ultimately found that the above SAC preparation is efficient in prevention, treatment or improvement of the degenerative bone disease such as osteoporosis or cancer. Accordingly, the present invention has been completed on the basis of the above finding.

### REFERENCES

### Patent Document

(Patent Document 1) Korean Patent Laid-Open Publication No. 10-2014-0064262

### SUMMARY OF THE INVENTION

An object of the present invention is to provide a composition for prevention, treatment or improvement of calcium-related degenerative disease, osteoporosis or cancers, which includes Sigma Anti-bonding Calcium (SAC) gluconate with physiologically activated ionic calcium.

To achieve the above object, the present invention provides a pharmaceutical composition for preventing or treating calcium-related degenerative diseases, osteoporosis or cancers, including Sigma Anti-bonding Calcium (SAC) gluconate formed by combining Sigma Anti-bonding Molecule Calcium Carbonate (SAC) with glucose, as an active ingredient.

In one embodiment of the present invention, the composition may include the SAC gluconate in an amount of 0.1 to 30% by weight (“wt. %”) based on a total weight of the composition.

In one embodiment of the present invention, the SAC may have a constitutional composition of: 98 to 99 wt. % of calcium carbonate (CaCO₃); and 1 to 2 wt. % of any one or more selected from the group consisting of Na₂O, MgO, SiO₂, Fe₂O₃, K₂O, MnO₂, TiO₂, Al₂O₃, Cl, S, P, Cu and Ni.

In one embodiment of the present invention, the SAC may be extracted from oyster shells and/or corals.

In one embodiment of the present invention, the composition may increase a concentration of 17β-estradiol in blood.

In one embodiment of the present invention, the composition may decrease concentrations of osteocalcin and CTx in blood.

In one embodiment of the present invention, the composition may increase a bone mineral density (BMD), and concentrations of calcium and phosphorous.

In one embodiment of the present invention, the calcium-related degenerative disease may be osteoporosis.

In one embodiment of the present invention, the calcium-related degenerative disease may be derived by ovariectomy or menopausal.

In one embodiment of the present invention, the cancer may be one or more selected from colorectal cancer, rectal cancer, breast cancer, prostate cancer, pancreatic cancer, brain tumor, lung cancer, liver cancer, stomach cancer, bone cancer, lymphatic cancer, bone marrow cancer and hematologic malignancy.

In addition, to achieve the above object, the present invention provides a health functional food composition for preventing or improving calcium-related degenerative diseases, osteoporosis or cancers, including Sigma Anti-bonding Calcium (SAC) gluconate formed by combining Sigma Anti-bonding Molecule Calcium Carbonate (SAC) with glucose, as an active ingredient.

Further, to achieve the above object, the present invention provides an animal feed composition for preventing or improving calcium-related degenerative diseases, osteoporosis or cancers, including Sigma Anti-bonding Calcium (SAC) gluconate formed by combining Sigma Anti-bonding Molecule Calcium Carbonate (SAC) with glucose, as an active ingredient.

The composition for preventing, treating or improving calcium-related degenerative diseases, osteoporosis or cancers of the present invention, which includes SAC gluconate as described above, may recover blood concentrations of 17 β-estradiol, osteocalcin and CTx as bone turnover markers to normal levels, and may increase the bone mineral density, as well as the calcium and phosphorus concentrations.

Therefore, the SAC gluconate-containing composition of the present invention is useful for a pharmaceutical composition for prevention or treatment of calcium-related degenerative diseases, osteoporosis or cancers, a health functional food composition for prevention or improvement of the above diseases, a livestock feed composition or the like. Further, the SAC gluconate described above enters the cells through glucose transport rather than a calcium channel in the cell, therefore, may accomplish an advantage of leading to the collapse of intracellular calcium homeostasis thus to allow the death of tumor cells.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates a functional mechanism of SAC carbonate used for preparation of Sigma Anti-bonding Calcium gluconate included in the composition of the present invention, wherein SAC (left) has a loose bond to other atom, radical, etc. compared to regular calcium phosphate (right), thus to easily enter the body;

FIG. 2 illustrates a bonding structure of SAC carbonate used for preparation of Sigma Anti-bonding Calcium gluconate included in the composition of the present invention, wherein the SAC has a loose sigma bonding, whereas regular calcium carbonate shows strong π(pi) bonding as calcium and oxygen in calcium carbonate are typically configured such that 2 electrons in 4S² orbital of calcium, that is, in the outermost shell of the carbon atom are bound to 6 electrons in 2P⁶ orbital of oxygen, that is, in the outermost shell of the oxygen atom through ionic bond;

FIG. 3 shows a chemical formula of the SAC gluconate;

FIGS. 4 and 5 show tumor growth graphs of the control rats;

FIG. 6 shows experimental results of animals with multiple myeloma;

FIG. 7 shows survivals of the control (bold line) and the experimental group (dotted line) as a result of multiple myeloma animal experiments; and

FIG. 8 illustrates delayed progress of multiple myeloma by SAC gluconate treatment in 5TGM1-grafted NSG mice.

### DETAILED DESCRIPTION OF THE INVENTION

The present invention relates to a composition for prevention, treatment or improvement of calcium-related degenerative diseases, osteoporosis or cancers, which includes Sigma Anti-bonding Molecule Calcium (SAC) gluconate prepared by combining SAC carbonate with glucose as an active ingredient.

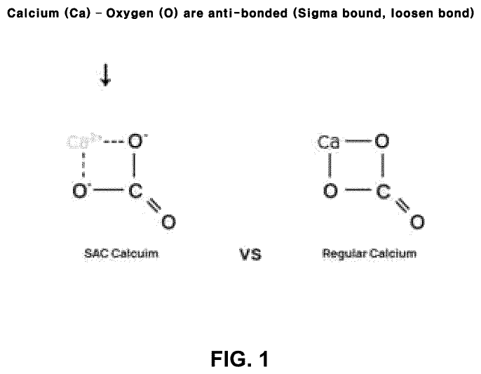

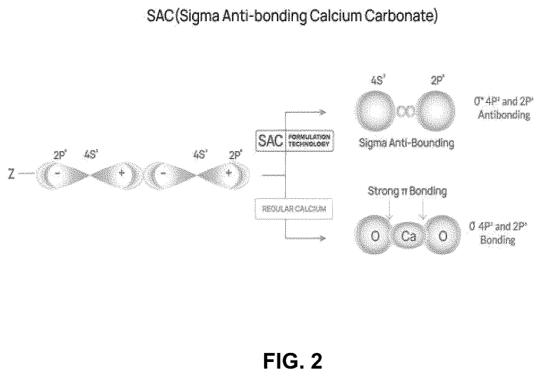

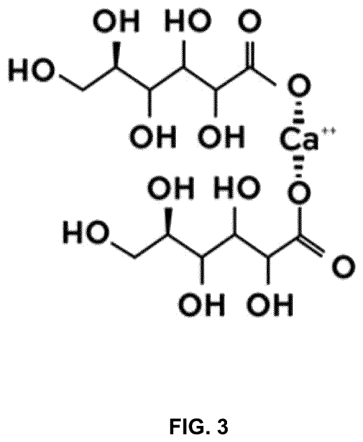

As shown in FIG. 1, oxygen-bound calcium molecules are very strongly bound and have low solubility, therefore, are hardly absorbed (or move) without the help of carrier proteins from vitamin D when the molecules are absorbed in the intestine.

On the other hand, SAC is anti-bounded and has higher ionization rate of 200 times or more in water, therefore, is easily absorbed (moves) into blood (passive transport mechanism) even without a digestion process after dosing.

Osteoporosis is one of the diseases under studies by numerous health professionals and nutritionists. In order to diagnose the osteoporosis, two types of methods including radiology and a biochemical method are mostly used. Compared to the radiology used for static analysis of bone conditions, biochemical diagnosis can analyze dynamic status of the bone by comparing inter-relationship between bone markers (J Biol Chem 1981; 256: 12760-12766).

As a result of investigating a variation in body weights of rats with ovariectomy (OVX) in the previous researches, the OVX group showed considerably high increase in the body weight compared to the control group (sham) (Bone (1997) 20: 55-61; Calcif Tissue Int (1997) 61(4): 336-344; Calcif Tissue Int (2002) 71(1): 69-79). Although the body weight is decreased due to stress for first 1 to 2 days after surgical operation, thereafter, the body weight is increased for 12 weeks or more (Physiol Gehav (1971) 7(6): 847-851; and Acta Endocrinologica (1973) 72: 551-568)). Similar to these study results, when the present inventors removed both ovaries from each of the experimental rats, the rats with ovariectomy showed quite increased body weight.

Meanwhile, there are continuous attempts to confirm kinetics of calcium metabolism using morphologies of bone tissues and isotopes of calcium. However, due to complication of experiments and clinical limitation, the necessity for a biological marker having high sensitivity and high applicable ability is further gradually increased so as to measure bone turnover associated with osteoporosis. For assessment of formation and resorption of bone matrix, activity of a basic or acidic oxidative enzyme (“oxidase”) of osteoblasts an osteoclasts. Further, there is an additional method for measuring bone matrix components that are secreted and then circulating in a system during bone formation and resorption processes.

Alkaline phosphatase (ALP) is increased within the serum according to the activity of osteoblasts. ALP in the serum shows positive interrelation to other biological markers, while having negative interrelation to bone mineral density, which in turn reflects a bone turnover rate increased due to bone loss after ovariectomy. However, since the bone turnover may also be increased by other diseases such as hyperthyroidism, there is a distorted aspect with respect to the correlation between the ALP and the osteoporosis.

According to Delmas's research (J Clin Endoclinol Metab (1983) 57: 1028-1030), ALP may become a marker of bone turnover. However, enzyme generation is not limited to the bone tissue (the liver generates isotonic enzyme of ALP, which reduces the sensitivity and specificity of the experiment), hence, is not desirable for bone loss. As a result of the experiment in the present invention, a concentration of enzyme in the serum (“serum enzyme concentration”) was increased but did not demonstrate the correlation with osteoporosis.

17β-estradiol, which is known as a compound closely relevant to bone metabolism, is mostly produced in the ovary, corpus luteum, placenta, adrenal and testicles and causes substantial changes during the menstrual cycle and a pregnancy period. Albright's research (Osteoporosis. Ann Intern Med (1947) 27(6): 861-882) has first introduced the fact that sex hormone deficiency possibly causes osteoporosis. Wroolie et al. (Am J Geriatr Psychiatry (2011) 19(9): 792-802) and Durador et al. (Rev Hosp Clin Fac Med Sao Paulo (997) 52(2): 60-62) have reported that ovariectomy-induced osteoporosis and menopausal would greatly reduce 17β-estradiol in blood, thereby to induce osteoporosis.

In the present invention, as compared to the control group, blood 17β-estradiol was considerably reduced in the experimental group, and the reduction of 17β-estradiol caused by ovariectomy was quite recovered by SAC treatment (Table 6). This is consistent with previous studies. The bone turnover rate was increased with the decrease of estrogen, and the bone loss was increased rapidly due to the increased bone resorption compared to bone formation.

Osteocalcin is present in bone and dentin and accounts for about 20% of non-collagenous protein in the bone. It has been reported that osteocalcin suppresses mineralization in vitro experiments, and the bone mineral density is increased in rats without osteocalcin gene (Drug Saf (2009) 32(3): 219-228).

A concentration of osteocalcin after post-menopausal is substantially increased due to rapid bone turnover, however, may be decreased with estrogen administration. Therefore, an increase in osteocalcin will improve the bone turnover (Delmas PD, et al. 1983). In the present invention, the concentration of osteocalcin was increased after ovariectomy while the concentration was considerably reduced after SAC treatment. This indicates a change in bone turnover and demonstrates the possibility of SAC as an osteoporosis therapeutic agent.

According to studies for women patients with femoral fractures for 2 years, CTx standard was increased compared to a normal control (Endocr J (2005) 52(6): 667-674; Arq Bras Endocrinol Metabol (2010) 54(2): 186-199; and J Bone Miner Res (1996) 11: 337-349). Further, in the present invention, it was confirmed that CTx was considerably increased after ovariectomy, but was then quite reduced by treatment with the SAC composition.

Low bone mineral density is a risk factor for bone fracture, which tends to develop to reduction in bone mineral density of the femoral region after post-menopausal (Bone (1997) 20: 55-61; and BMJ (1996) 312: 1254-1259]). In the present invention, the bone mineral density of OVX group was lower than that of Sham group, and treatment using SAC gluconate composition after ovariectomy improved BMD.

Increase of calcium extrusion from the body skeleton is a change appeared first by estrogen deficiency. Due to rapid bone loss, the secretion of parathyroid hormone (“parathormone”) is decreased, thereby leading to a decrease in intestinal calcium absorption and ultimately calcium loss in the human body as well as bones (Clin Exp Obstet Gynecol (2004) 31(4): 251-255; and Ann N Y Acad Sci (1998) 854: 336-351).

According to recent research results for investigation of influences of calcium supplement on the bone mineral density and bone fractures in menopausal women, it could be seen that calcium administration for 2 years or more is effective in reduction of bone loss in placebo group (Ther Clin Risk Manag (2011) 7: 157-166; and Drug Saf (2009) 32(3): 219-228). In the present invention, it was confirmed that, as compared to Sham group, OVX group showed quite reduction of calcium while OVX+SAC group was not significantly different from the Sham group. This indicates that the progress to osteoporosis is reduced. That is, the osteoporosis derived from ovariectomy or menopausal may be reduced by treatment using SAC gluconate composition.

Accordingly, the present invention provides a composition for preventing, treating or improving osteoporosis, which includes SAC gluconate as an active ingredient. As used herein, the “prevention” means all procedures to inhibit the formation of osteoporosis or delay the progress thereof by administration of the inventive composition. Further, the “treatment” and “improvement” mean all actions to improve or beneficially alter symptoms of osteoporosis by administration of the inventive composition.

In order to prevent or terminate tumor cell proliferation, the present invention has been proposed to provide a novel material for death of tumor cells, characterized in that: osteoclasts are inactivated by increasing calcitonin which provides calcium from an outside, thus to block calcium supply to the tumor cells; other than calcium fed through a calcium channel, SAC gluconate provides calcium through glucose transport thus to break down calcium homeostasis; calcium, of which excessively supplied calcium ions are attached to glucose within the tumor cell lacking oxygen, generates free radicals during glycolysis, thus to oxidize and destruct cellular lipids.

In normal cells rich in oxygen, calcium ions introduced through glucose transport may enable normal metabolism. The glucose used herein may include both of L-type and D-type glucoses.

Activated ionic calcium of SAC used for preparation of SAC gluconate in the present invention is produced by treating calcium carbonate through a physical and/or magnetic process so that Sigma Anti-bonding Molecule Calcium Carbonate (SAC) with weak bonding force between calcium and a carbonyl radical, that is, physiological activated ionic calcium may produce calcium carbonate having a weak bonding force, wherein the above calcium has a weak bonding force to other anionic atoms or radicals.

Accordingly, the calcium described above may be easily absorbed into cells without 1,2,5-dihydroxycholecalciferol (activated form of vitamin D) (“passive transport”), and may increase ionic calcium in blood thus to promote cell and bone metabolism. Further, the SAC may help hormone and enzyme activation, and effectively supply calcium into cells requiring metabolism, thereby activating endogenous metabolism.

According to an embodiment of the present invention, SAC used in the SAC gluconate composition of the present invention may be prepared using shells of shellfish including oysters, corals, etc.

With regard to the preparation of SAC and SAC gluconate, as well as the production of SAC gluconate composition, physical and/or magnetic processes may be applied. For example, the SAC and SAC gluconate composition may be produced by a method including the following steps of: (a) washing and crushing oyster shells to a size of 250 meshes of more; (b) drying the crushed oyster shells through heating at a low temperature; (c) super-heating the low-temperature heated oyster shells at 1400° C. for 24 hours; (d) cooling the heated shells for 24 hours; (e) cooling the shells to −60° C. under nitrogen gas; (f) injecting D-glucose or L-glucose and then injecting pure carbon dioxide gas therein; (g) beating the product to room temperature; and (h) injecting ultrapure water into the product.

In one embodiment of the present invention, the SAC gluconate composition of the present invention preferably includes physiologically activated ionic calcium, which is specifically designed to facilitate in vivo absorption (migration). The SAC gluconate may have high ionization degree if needed.

In one embodiment of the present invention, a concentration of the SAC compound contained in the SAC gluconate composition of the present invention is 1200 ppm or more in terms of calcium. SAC gluconate may range from 10 to 30 wt. % based on a total weight of the composition, but it is not limited thereto.

The composition of the present invention may contain various minerals as well as calcium ions. In one embodiment of the present invention, the calcium composition may be prepared in the form of an emulsion for oral administration.

When the composition of the present invention is used as a pharmaceutical composition, it may further include an active ingredient exhibiting the same or similar function. The composition of the present invention can be clinically administered orally or parenterally, and can be used in the form of a general pharmaceutical preparation.

When the composition of the present invention is formulated, it may be prepared using diluents or excipients such as fillers, extenders, binders, humectants, disintegrants, surfactants, etc., which are usually used. The solid preparations for oral administration include tablets, pills, powder, granules, capsules and the like. These solid preparations can be prepared by mixing at least one excipient such as starch, calcium carbonate, sucrose or lactose, gelatin and the like. In addition to simple excipients, lubricants such as magnesium stearate talc may also be used. Examples of the liquid preparation for oral administration may include suspensions, oral liquids, emulsions, syrups and the like. In addition to water and liquid paraffin as simple diluents commonly used, various excipients such as wetting agents, sweeteners, fragrances, and the like may be included.

Formulations for parenteral administration may include sterilized aqueous solutions, non-aqueous solvents, suspensions, emulsions, freeze-dried preparations, and suppositories. In the case of the non-aqueous solvent or suspending solvent, propylene glycol, polyethylene glycol, vegetable oil such as olive oil, injectable ester such as ethyl oleate, and the like, may be used. As a base of the suppository, witepsol, macrogol, tween 61, cacao butter, laurin, glycerogelatin, and the like may be used.

An administration amount, that is, dosage of the composition of the present invention may vary depending on the patient's body weight, age, sex, health condition, diet, administration time, administration method, excretion rate, and disease severity. Specifically, a daily dosage may be 80 mcg/kg, preferably 80 to 160 mcg/kg in terms of calcium amount, and may be administered 1 to 6 times a day. The SAC gluconate of the present invention may be used alone or in combination with methods using surgery, radiation therapy, hormone therapy, chemotherapy and biological response modifiers.

For various purposes of the SAC gluconate including edible use, medicine or livestock administration, etc., in order to provide SAC calcium to the human body and livestock products, it is preferable to produce the product that facilitates in vivo absorption (migration) by reducing a bonding force of calcium carbonate molecules. That is, the SAC gluconate is excellent in in vivo absorbability, can increase the concentration of 17β-estradiol in blood while decreasing the concentration of osteocalcin and CTx concentration. Further, by increasing the bone mineral density, calcium and phosphorous concentrations, the above SAC gluconate may be usefully applied as an active ingredient for a health functional food as well as for prevention and improvement of degenerative diseases such as osteoporosis, cancer, etc.

When the SAC gluconate composition is used as a food additive, the SAC gluconate may be added as it is or in combination with other food ingredients. A mixing amount of the active ingredients may be appropriately determined depending on the purpose of use thereof. Generally, the SAC gluconate may be added in an amount of not more than 1 part by weight (“wt. part”), preferably 1 wt. part or less, based on the whole raw material, but it is not limited thereto. In the case of long term intake intended for health or hygiene purposes or for the purpose of controlling health, the above amount may be the above range or less. Further, since there is no problem in terms of safety, the active ingredient may be used in an amount exceeding the above range.

There is no particular limitation on the type of food. Examples of the food to which the above substance can be added may include meat, sausage, bread, chocolate, candy, snack, confectionery, pizza, ramen, other noodles, gums, dairy products including ice cream, various soups, beverage, tea, hangover drinks, alcoholic beverage such as wine, whisky, etc., vitamin complexes, or the like, and substantially all healthy foods in a conventional sense.

The health beverage composition may further contain various flavors or natural carbohydrates as additional ingredients, like any conventional beverage. Such natural carbohydrates may be, for example, monosaccharides such as glucose and fructose, disaccharides such as maltose and sucrose, polysaccharides such as dextrin and cyclodextrin, and sugar alcohols such as xylitol, sorbitol and erythritol.

Examples of sweeteners used herein may include natural sweeteners such as thaumatin and stevia extract, synthetic sweeteners such as saccharin and aspartame and the like. A proportion of the natural carbohydrate is generally about 0.01 to 0.04 g, and preferably about 0.02 to 0.03 g per 100 ml of the composition of the present invention. Further, the composition of the present invention may further include a variety of nutrients, vitamins, electrolytes, flavors, colorants, pectic acid and salts thereof, alginic acid and salts thereof, organic acids, protective colloid thickeners, pH adjusters, stabilizers, preservatives, glycerin, carbonation agents used in beverages and the like. In addition, the composition of the present invention may contain fruit flesh for the production of natural fruit juices, fruit juice drinks and vegetable drinks.

These components may be used independently or in combination. A proportion of such additives is not critical, but is generally selected in the range of 0.01 to 0.1 wt. parts per 100 wt. parts of the composition of the present invention.

The present invention also provides a method for treatment of degenerative bone diseases or cancers, which includes administering a pharmaceutically effective amount of the composition to an individual. The term “pharmaceutically effective amount” means an amount sufficient to treat a disease at a reasonable benefit or risk ratio applicable to medical treatment, which may be determined by the type of disease, severity, activity of the drug, sensitivity to the drug, administration time, administration route, excretion rate, duration of treatment, factors including concurrently useable drugs, and other factors well known in the medical field.

The term “individual” means any animal such as a human, a monkey, a dog, a goat, a pig, or a mouse having a disease in which symptoms of osteoporosis can be improved by administering the composition of the present invention. The composition may contain at least one active ingredient which exhibits the same or similar functions in addition to the SAC gluconate of the present invention.

The composition is possibly administered orally or parenterally at the time of clinical administration and may be used in the form of a general pharmaceutical preparation. The dosage of the composition may vary depending on the patient's body weight, age, sex, health condition, diet, administration time, administration method, excretion rate, and disease severity. The methods may be used alone or in combination with methods using surgery, radiation therapy, hormone therapy, chemotherapy, and biological response modifiers.

Hereinafter, the present invention will be described in detail with reference to examples. However, these examples are intended to further illustrate the present invention, and the scope of the present invention is not limited to these examples.

### <Example 1> Experimental Material

### 1. Composition

The constitutional composition of Sigma Anti-bonding Molecule Carbonate (SAC; purity 98%) is shown in Table 1 below, and the SAC has a specific gravity of 7.0 to 7.5 at 20° C. and heavy metal content (As, Pb, Cd and Hg) of less than 1 ppm. Further, the specific gravity of the SAC gluconate dissolved in water ranges from 1.16 to 1.20.

TABLE 1

| Contents | Concentration (weight %) |
|---|---|
| CACO₃ | 98.5 |
| Na₂O | 0.56 |
| MgO | 0.49 |
| SiO₂ | 0.22 |
| Fe₂O₃ | 0.02 |
| K₂O | 0.04 |
| MnO₂ | 0.001 |
| TiO₂ | 0.002 |
| Al₂O₃ | 0.012 |
| Cl | 0.02 |
| S | 0.09 |
| P | trace |
| Cu | trace |
| Ni | trace |

### 2. Experimental Animal

12 week old female SPF Sprague-Dawley rats (body weight: 250 g; n=15) were purchased from the Korean Laboratory Animals Center (Eumseong, South Korea). Breeding conditions were as follows: temperature, 23+3° C.; relative humidity, 55+10%; lighting time, 12 hours (08:00-20:00); ventilation frequency, 10-20 changes/h; illumination, 150-300 lux. The temperature and humidity were measured using a hygrometer in units of time, and there were no variables that could affect the experimental results. The animals were fed normal rodent feed and purified water.

### <Example 2> Weight Change and Feeding Observation in Experimental Animals

### 1. Classification and SAC Gluconate Treatment

The experimental animals were randomly divided into three groups. After adaptation, bilateral laparotomy (sham) or bilateral ovariectomy (OVX) was performed on the rats. At 3 weeks after surgery and recovery, the above OVX rats were randomly divided into three groups: vehicle-treated (OVX), SAC-treated (OVX+SAC), and SAC gluconate-treated (OVX+SAC gluconate) groups.

Only OVX+SAC and OVX+SAC gluconate rats were allowed to drink water containing 0.0012% SAC gluconate for 3 to 12 weeks after surgery. Changes in the body weight and feeding and water intake were measured at a regular interval.

### 2. Change in Body Weight and Feeding

Post-operative stress-induced weight loss was observed in all of the OVX, OVX+SAC and OVX+SAC gluconate groups till day 2 after surgery, however, there was no further increase in the body weight and feed intake since then. Weight gain after ovariectomy was slightly higher in the ovariectomy group than in the Sham group (Table 2 below). Although no statistical significance was observed, SAC gluconate treatment increased body weight.

TABLE 2

| | Body | | |
|---|---|---|---|
| Group | weight gain | Food intake | |
| (5 rats/group) | (g/day | (g/day ) | FER |
| Sham | 0.84 ± 3.76 | 19.15 ± 0.98^{a} | 0.04 ± 3.8 |
| OVX | 0.98 ± 6.06 | 20.53 ± 0.81b^{b} | 0.04 ± 7.48 |
| OVX + SAC | 2.38 ± 6.74 | 21.59 ± 1.39^{b} | 0.11 ± 4.84 |
| OVX + SAC Gluconate | 2.82 ± 6.74 | 25.59 ± 1.39^{b} | 0.11 ± 4.84 |

Values are mean ± SD for 5 rats. Means with different superscript letters are significantly different at p < 0.05 by Duncan's multiple range tests. Sham: sham operation; OVX: no-treatment after ovariectomy; OVC + SAC: sigma anti-bonding calcium treatment for 12 weeks after ovariectomy; OVX + SAC gluconate: sigma anti-bonding calcium gluconate treatment after ovariectomy.

With respect to feed intake, a significant change (P<0.05) of daily feeding was observed in the OVX group compared to the Sham group. Further, SAC and SAC gluconate further increased feed intake, thereby resulting in increased feed efficiency ratio (FER). The difference in feed intake according to SAC or SAC gluconate treatment seemed to have affected weight change. On the other hand, water intake was not different between the two groups.

### 3. Change in Weight of Organ

The weights of different organs are shown in Table 3 below.

TABLE 3

| | Group | | | |
|---|---|---|---|---|
| | | | | OVX + SAC |
| Organ (unit) | Sham | OVX | OVX + SAC | gluconate |
| Body weight (kg) | 310.84 ± 11.14 | 353.75 ± 10.18 | 371.36 ± 9.97 | 426265 ± 9.97 |
| Uterus(g) | 0.1889 ± 0.05 | 0.0381 ± 0.01 | 0.0444 ± 0.02 | 0.0524 ± 0.02 |
| Vagina(g) | 0.1128 ± 0.03 | 0.0501 ± 0.02 | 0.0575 ± 0.02 | 0.0665 ± 0.02 |
| Liver (g) | 2.3435 ± 0.15 | 2.1675 ± 0.10 | 2.1820 ± 0.15 | 2.5220 ± 0.15 |

Kidney:

| | | | | |
|---|---|---|---|---|
| Right (g) | 0.3236 ± 0.02 | 0.2682 ± 0.00 | 0.2626 ± 0.02 | 0.2926 ± 0.02 |
| Left (g) | 0.3289 ± 0.03 | 0.2712 ± 0.01 | 0.2687 ± 0.01 | 0.2977 ± 0.01 |
| Spleen (g) | 0.1840 ± 0.02 | 0.1692 ± 0.02 | 0.1856 ± 0.02 | 0.2056 ± 0.02 |

Adrenal gland

| | | | | |
|---|---|---|---|---|
| Right (g) | 0.0127 ± 0.0024 | 0.0093 ± 0.0012 | 0.0094 ± 0.0014 | 0.0098 ± 0.0014 |
| Left (g) | 0.0128 ± 0.0020 | 0.0092 ± 0.0008 | 0.0094 ± 0.0014 | 0.0098 ± 0.0014 |
| Brain | 0.65550 ± 0.03 | 0.6044 ± 0.03 | 0.5526 ± 0.02 | 0.6126 ± 0.02 |
| Pituitary (mg) | 0.0055 ± 0.00005 | 0.0038 ± 0.0002 | 0.0042 ± 0.0010 | 0.0043 ± 0.0010 |

Femur

| | | | | |
|---|---|---|---|---|
| Right (g) | 0.3296 ± 0.02 | 0.2960 ± 0.02 | 0.2902 ± 0.02 | 0.3302 ± 0.02 |
| Left (g) | 0.3524 ± 0.03 | 0.2930 ± 0.01 | 0.2935 ± 0.02 | 0.3335 ± 0.02 |

Values are mean ± SD for 5 rats. Sham: sham operation; OVX: no-treatment after ovariectomy; OVC + SAC: sigma anti-bonding calcium treatment for 12 weeks after ovariectomy; OVX + SAC gluconate: sigma anti-bonding calcium gluconate treatment for 12 weeks after ovariectomy.

The weights of the reproductive organs (uterus and vagina) were significantly reduced by ovariectomy and were not restored by the SAC gluconate. The weights of other organs including the femur were slightly decreased after ovariectomy, but did not show significant changes by SAC gluconate treatment. Experimental results show that the SAC gluconate does not significantly affect the weights of the organs.

### <Example 3> Hematology and Blood Biochemistry

### 1. Blood Collection

Blood samples were collected at 12 weeks post-surgery. Animals were deprived of food for 16 hours and then anesthetized prior to blood sampling (Zoletile 50, Virbac, Korea). 0.5 mL of blood was collected from the abdominal artery and stored at 4° C. in a refrigerator.

A portion of the blood was used for hematological experiments, and the remaining blood was centrifuged at 3,000 rpm for 10 minutes to obtain serum. The serum was used for blood biochemical experiments and bone metabolism markers 17β-estradiol, osteocalcin and CTx analysis.

### 2. Hematology and Blood Biochemical Analysis Method

Hematological experiments were performed using T-540 Coulter Counter (Coulter Electronics Inc., Hialeah, USA) and ADVIA 120 Hematology System (Bayer Healthcare LLC, Tarrytown, USA). White blood cells (WBCs), differential leukocyte counts, hematocrit, red blood cells (RBCs), mean corpuscular volume (MCV), mean corpuscular hemoglobin (MCH), mean corpuscular hemoglobin concentration (MCHC), and platelet count were subjected to analysis, respectively.

The parameters measured for blood biochemical analysis (Ciba-Corning 644 Na/K/Cl Analyzer, Ciba-Corning, Medfield, USA) were as follows: alanine aminotransferase (ALT), aspartate aminotransferase (AST), alkaline phosphatase (ALP), blood urea nitrogen (BUN), glucose, triglycerides (TG), total cholesterol (TC), high-density lipoproteins (HDL), low-density lipoproteins (LDL), total proteins (TP), albumin, calcium and phosphorus. Further, a concentration of serum 17β-estradiol as a parameter of bone metabolism was analyzed using a 17β-estradiol kit (Diagnostic Products Corporation, Los Angeles, USA).

Osteocalcin levels were measured using a sandwich ELISA kit (Biomedical Technologies Inc., Stoughton, USA), while CTx levels were measured using a serum RatLaps ELISA kit (IDS Inc., Fountain Hills, USA).

### 3. Hematology and Blood Biochemical Analysis Results

As a result of hematological analysis, no significant variation was found, however, it was observed that eosinophils were significantly increased in the SAC gluconate treated group after ovariectomy (Table 4 below).

TABLE 4

| | Group | | | |
|---|---|---|---|---|
| | | | | OVX + SAC |
| Parameters | Sham | OVX | OVX + SAC: | gluconate: |
| White blood cell (10³/μl) | 4.95 ± 3.15 | 4.69 ± 1.79 | 4.84 ± 1.17 | 4.94 ± 2.17 |
| Neutrophils (%) | 22.28 ± 4.48 | 23.4 ± 7.18 | 21.21 ± 6.19 | 22.21 ± 5.19 |
| Eosinophils (%) | 1.0 ± 0.52 | 1.16 ± 0.27 | 5.04 ± 3.88 | 5.06 ± 2.88 |
| Basophils (9%) | 0.04 ± 0.05 | 0.04 ± 0.05 | 0.02 ± 0.04 | 0.03 ± 0.04 |
| Lymphocytes (%) | 73.72 ± 3.97 | 72.54 ± 6.96 | 70.36 ± 8.78 | 73.36 ± 3.78 |
| Monocytes (%) | 2.68 ± 0.78 | 2.58 ± 0.33 | 2.50 ± 1.44 | 2.60 ± 1.04 |
| Red blood cell (10³/μl) | 7.95 ± 0.64 | 8.26 ± 0.24 | 8.23 ± 0.55 | 8.33 ± 0.45 |
| Hematocrit (%) | 44.16 ± 2.47 | 45.30 ± 1.23 | 43.94 ± 2.18 | 44.94 ± 1.18 |
| Mean corpuscular volume (fl) | 55.68 ± 2.11 | 54.82 ± 0.82 | 53.44 ± 1.24 | 54.44 ± 1.24 |
| Mean corpuscular hemoglobin (pg) | 32.26 ± 1.36 | 30.94 ± 0.91 | 30.50 ± 0.75 | 32.50 ± 1.75 |
| Mean corpuscular hemoglobin | 57.94 ± 1.88 | 56.40 ± 1.33 | 57.14 ± 0.76 | 57.54 ± 1.76 |
| concentration (g/dl) | | | | |
| Platelet (10³/mm³) | 1440.20 ± 153.83 | 1234.80 ± 82.35 | 1297.0 ± 91.27 | 1397.0 ± 81.27 |

Values are mean ± SD for 5 rats. Sham: sham operation; OVX: no-treatment after ovariectomy; OVC + SAC: sigma anti-bonding calcium treatment for 12 weeks after ovariectomy; OVX + SAC gluconate: sigma anti-bonding calcium gluconate treatment for 12 weeks after ovariectomy.

With regard to blood parameters in relation to hepatotoxicity (AST, ALT and ALP), hepatic energy storage and pancreatic damage (glucose), hepatic lipid metabolism (triglyceride, total cholesterol, DHL and LDL), liver protein synthesis (total protein and albumin), renal damage and electrolyte balance (BUN, calcium, and phosphorus), no significant change was observed (Table 5 below).

TABLE 5

| | Rat | | | |
|---|---|---|---|---|
| | | | | OVX + SAC |
| Items | Sham | OVX | OVX + SAC | gluconate |
| GOT (IU/l) | 159.8 ± 37.8 | 163.0 ± 20.3 | 148.0 ± 30.4 | 158.0 ± 32.4 |
| GPT (IU/l) | 19.2 ± 4.5 | 18.6 ± 4.3 | 22.4 ± 4.0 | 20.4 ± 4.5 |
| ALP (IU/l) | 108.6 ± 29.7 | 112.6 ± 17.9 | 108.2 ± 20.7 | 108.5 ± 21.7 |
| BUN (mg/dl) | 9.7 ± 2.3 | 10.2 ± 2.4 | 9.2 ± 2.4 | 9.5 ± 2.3 |
| Glucose (mg/dl) | 109.8 ± 7.4 | 115.0 ± 17.7 | 112.4 ± 7.7 | 110.4 ± 8.7 |
| TG (mg/dl) | 11.8 ± 6.1 | 19.8 ± 6.6 | 20.6 ± 7.7 | 18.6 ± 6.7 |
| TC (mg/dl): | 59.6 ± 4.8 | 60.2 ± 24.3 | 73.0 ± 17.3 | 63.0 ± 15.3 |
| HDL(mg/dl) | 20.8 ± 2.5 | 19.8 ± 6.8 | 23.8 ± 5.2 | 22.8 ± 6.2 |
| LDL-C(mg/dl) | 10.2 ± 4.1 | 8.9 ± 5.3 | 8.0 ± 5.2 | 8.1 ± 4.2 |
| TP (mg/dl) | 5.0 ± 0.2 | 4.7 ± 0.2 | 4.7 ± 0.2 | 4.9 ± 0.2 |
| Albumin (mg/dl) | 3.0 ± 0.1 | 2.7 ± 0.1 | 2.7 ± 0.1 | 2.8 ± 0.1 |
| Phosphorus (mg/dl) | 4.4 ± 0.4 | 4.3 ± 0.3 | 4.1 ± 0.6 | 4.2 ± 0.3 |
| Calcium (mg/dl) | 6.8 ± 0.3 | 6.7 ± 0.2 | 6.7 ± 0.4 | 6.8 ± 0.3 |

GOT: glutamic oxaloacetic transaminase; GPT: glutamic pyruvic transaminase; BUN: blood urea nitrogen; ALP: alkaline phosphatase; TG: triglyceride; TC: total cholesterol; HDL: high density lipoprotein; LDL-C: low density lipoprotein; TP: total protein. Values are mean ± SD for 5 rats. Sham: sham operation; OVX: no-treatment after ovariectomy; OVC + SAC: sigma anti-bonding calcium treatment for 12 weeks after ovariectomy; OVX + SAC gluconate: sigma anti-bonding calcium gluconate treatment for 12 weeks after ovariectomy.

However, in particular, the bone growth marker ALP with negative correlation to the bone mineral density was recovered to a normal level in SAC-treated animals and SAC gluconate-treated animals.

### 4. Blood Markers of Bone Formation

As shown in values of bone metabolism index test in Table 6 below, blood 17β-estradiol concentration was significantly reduced by ovariectomy, while the reduction of 17β-estradiol by ovariectomy was quite recovered by the SAC gluconate treatment. Further, as a bone resorption marker with negative correlation to the bone mineral density, C-terminal telopeptide was also reduced thus to indirectly determine an increase in the bone mineral density.

TABLE 6

| | Estradiol | Osteocalcin) | C-terminal telopeptide |
|---|---|---|---|
| Group | (pg/ml) | (ng/mL) | (ng/ml) |
| Sham | 4.536 ± 0.343^{a} | 3.398 ± 0.294^{a} | 4.751 ± 0.244^{a} |
| OVX | 3.920 ± 0.277^{a} | 4.047 ± 0.501b^{b} | 5.082 ± 0.462^{ab} |
| OVX + SAC | 4.217 ± 0.247^{a} | 3.558 ± 0.154^{ac} | 4.360 ± 0.495^{a} |
| OVX + SAC Gluconate | 4.417 ± 0.347^{a} | 3.458 ± 0.254^{ac} | 4.340 ± 0.395^{a} |

Values are mean ± SD for 5 rats. Means with different superscript letters are significantly different at p < 0.05 by Duncan's multiple range tests. Sham: sham operation; OVX: no-treatment after ovariectomy; OVC + SAC: sigma anti-bonding calcium treatment for 12 weeks after ovariectomy; OVX + SAC gluconate: sigma anti-bonding calcium gluconate treatment for 12 weeks after ovariectomy.

The above results show that a decrease in the estrogen concentration may increase osteoclast formation and bone resorption, which can be inhibited by SAC gluconate. Increases in osteocalcin and CTx were observed in the ovariectomized (OVX) group compared to the Sham group. However, the increase of these metabolic factors was recovered to normal levels by the SAC gluconate treatment.

### <Example 4> Observation of Physical Property

### 1. Measurement of Bone Mineral Density and Mineral Content

Using dual energy X-ray absorptionmetry (DEXA; Prodigy Advance, Donga imaging, Korea), the bone mineral density (BMD) of the femur was measured. Immediately after BMD measurement, the femur was subjected to mechanical test at room temperature using a material testing machine (MZ500D; Maruto, Tokyo, Japan). For stabilization, the mid-diaphysis of the thigh was placed on two supports at an interval of 4 mm on a test device. A three-point bending test was performed in front and rear directions at the midpoint between the two supports.

Breaking force was measured using software (CTR win, Ver. 1.05) to measure bone strength. After measuring the breaking force, the thigh was dried in a muffle furnace at 120° C. for 6 hours and then the dry weight was recorded. Then, the thigh was placed at 800° C. for 16 hours, and the weight of the ash was measured.

100 mg of ash was then dissolved in 2 mL of 37% HCl and diluted with distilled water. Calcium and phosphorus contents were measured using an atomic absorption spectrophotometer (PerkinElmer; A Analyst 100 Spectrophotometer, Boston, USA).

### 2. Femoral Breaking Degree

The femoral breaking force of the OVX group was considerably reduced, thereby indicating a deterioration in bone integrity. Table 7 below shows BMD measurement values.

TABLE 7

| Group | BMD |
|---|---|
| Sham | 0.2276 ± 0.011^{a} |
| OVX | 0.1965 ± 0.012^{b} |
| OVX + SAC | 0.2276 ± 0.012^{a} |
| OVX + SAC Gluconate | 0.2376 ± 0.011^{a} |

Values are mean ± SD for 5 rats. Means with different superscript letters are significantly different at p < 0.05 by Duncan's multiple range tests. Sham: sham operation; OVX: no-treatment after ovariectomy; OVC + SAC: sigma anti-bonding calcium treatment for 12 weeks after ovariectomy; OVX + SAC gluconate: sigma anti-bonding calcium gluconate treatment for 12 weeks after ovariectomy.

Along with a change in breaking force, BMD as well as concentrations of ash, calcium and phosphorus were also reduced by ovariectomy. However, the breaking force and related components (BMD, calcium and phosphorus) were completely recovered by the SAC gluconate treatment.

### <Example 5> Rectal Cancer Animal Experiment

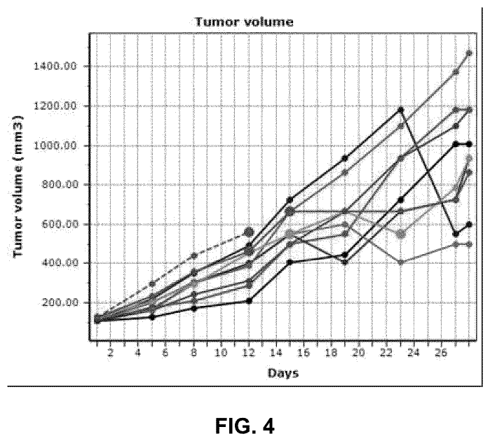

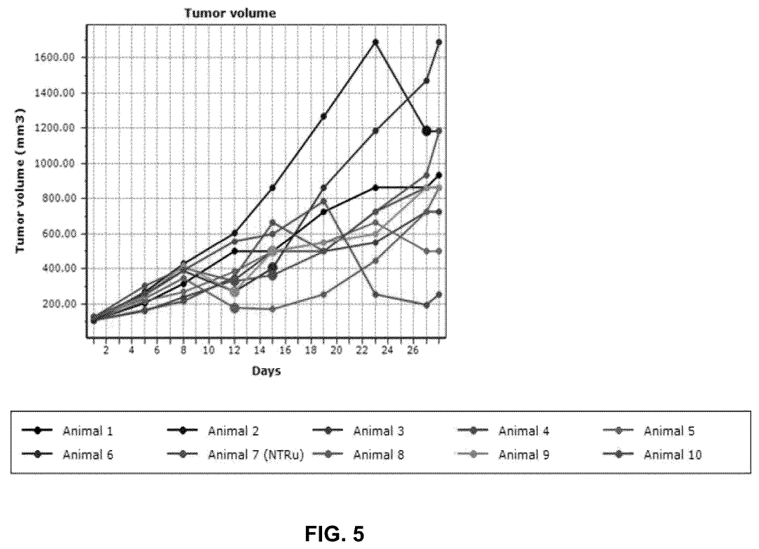

Using human colo 205 rectal cancer cells as xenograft model, clinical experiments were performed with adenocarcinoma nude mice for 28 days. Experimental results are shown in FIGS. 4 and 5. In this regard, the experimental method and usage are shown in Table 8 (Protocol Design for the Colo205-e347 Study) and Table 9 (Final treatment regimen at completion of the study) below.

TABLE 8

| | | Treatment Regimen | | | |
|---|---|---|---|---|---|
| Group | n | Agent | mg/kg | Route | Schedule |
| 1 | 10 | water | — | po | tid to end first Day 2 doses |
| 2 | 10 | SAC | 0.83 | po | bid to end first Day 2 dose |
| | | Calcium | with | | |
| | | | water | | |
| 3 | 10 | SAC | 0.83 | po | tid to end first Day 2 doses |
| | | Gluconate | | | |

TABLE 9

| | | Treatment Regimen | | | | MTV (n) | | Statistical | Regressions | | | Mean BW | Deaths | |
|---|---|---|---|---|---|---|---|---|---|---|---|---|---|---|
| Group | n | Agent | mg/kg | Route | Schedule | Day 28 | % TGI | Significance | PR | CR | TFS | Nadir | TR | NTR |
| 1 | 9 | vehicle | — | po | tid to end | 936 (9) | — | — | 0 | 0 | 0 | — | 0 | 1 |
| | | | | | first Day 2 doses | | | | | | | | | |
| 2 | 9 | SAC | 0.83 | po | bid to end | 896 (9) | 4 | ns | 0 | 0 | 0 | −0.4% Day 2 | 0 | 1 |
| | | Calcium | | | first Day 21 dose | | | | | | | | | |
| 3 | 10 | SAC | 0.83 | po | tid to end | 864 (10) | 8 | ns | — | — | — | — | 0 | 0 |
| | | Gluconate | | | first Day 2 doses | | | | | | | | | |

vehicle = DI water
Study Endpoint = 2000 mm³; Study Duration = 28 Days
n = number of animals in a group not dead from accidental or unknown causes, or euthanized for sampling
% TGI = [1 − (MTV_{drug treated}/MTV_{control})] × 100 = percent tumor growth inhibition, compared to Group 1
Statistical Significance (Mann-Whitney U test): ne = not evaluable; ns = not significant; * = P ≤ 0.05; ** = P ≤ 0.01; *** = P ≤ 0.001, compared to Group 1
MTV (n) = median tumor volume (mm³) for the number of animals no the Day of TGI analysis (includes animals with tumor volume at endpoint)
PR = partial regression; CR = complete regression; TFS = tumor free survivors, i.e., CRs at end of study
Mean BW Nadir = lowest group mean body weight, as % change from Day 1; — indicates no decrease in mean body weight was observed
TR = treatment-related death; NTR = non-treatment-related death

As shown in FIG. 4, in the case of the SAC gluconate dosing mouse, it could be seen that tumor growth was slowed down after 12 days. Further, as shown in FIG. 5, it could be seen that tumor growth became dull and a size of tumor was reduced after 2 or 3 weeks from the dose.

Finally, according to the comparison of tumor growth for 28 days, it was confirmed that the SAC gluconate group has 8% decreased tumor size compared to the control group, thereby indicating that SAC gluconate is effective in animal rectal cancer.

### <Example 6> Multiple Myeloma Animal Experiment

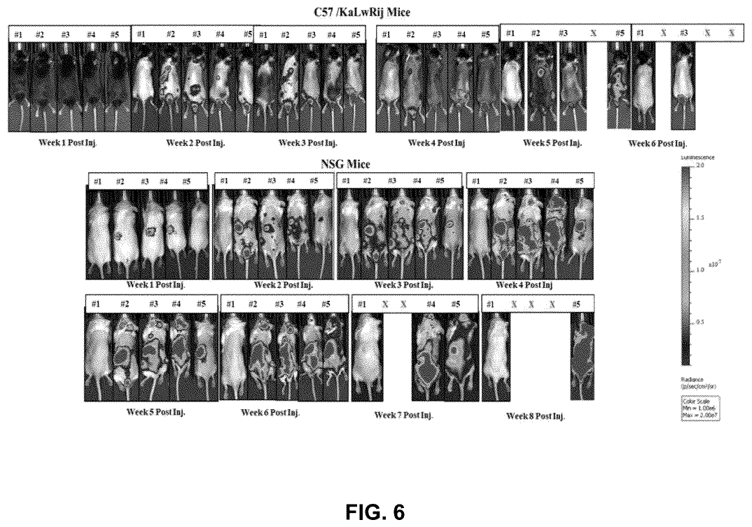

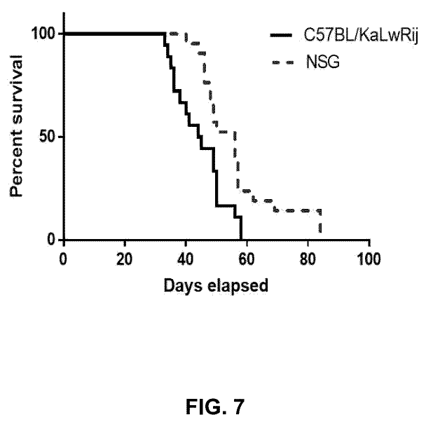

After injecting multiple myeloma (MM) to the experimental animals, 80 mg/kg of SAC gluconate was orally injected 3 times per day, followed by measuring survival days. The measured results are shown in FIGS. 6 and 7.

As shown in FIG. 6, as a result of injecting MM cell C57/KaLwRij to the experimental animal (top image), it could be seen that the tumor is grown over time, finally leading to death. Further, as shown in FIG. 7 that indicates comparison of survival periods between the control group (bold line) and the experimental group (dotted line), it could be seen that the control group survived for 58 days after tumor inoculation, while the SAC gluconate group (NSG) survived for 83 days after inoculation to tumor cells, thereby demonstrating 43% extension.

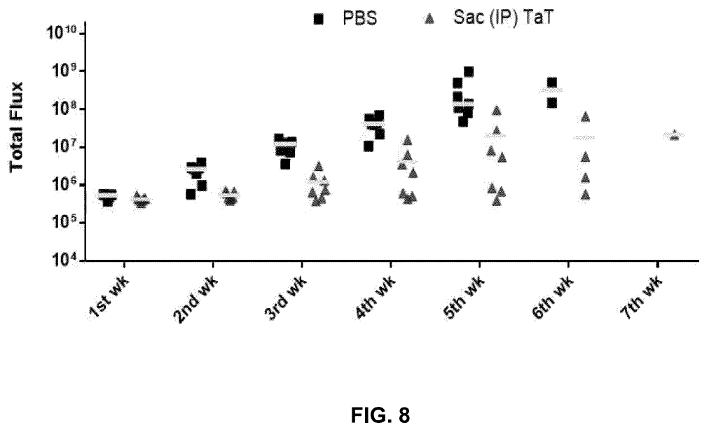

Further, as shown in FIG. 8, it could be seen that the tumor growth in the control group rats was continued, whereas the tumor growth in the SAC gluconate dosed rats was stopped or at least became dull.

As such, the present invention has been described with reference to the preferred embodiments. It will be understood by those skilled in the art that various changes or modifications of the present invention may be implemented without departing from the scope and essential features of the invention.

Therefore, the disclosed embodiments should be considered in an illustrative aspect rather than a restrictive sense. The scope of the present invention is defined by the appended claims rather than by the foregoing description, and all differences within the scope of equivalents thereof should be construed as being included in the present invention.

What is claimed is:

1. A pharmaceutical composition for preventing or treating calcium-related degenerative diseases, osteoporosis or cancers, comprising Sigma Anti-bonding Calcium (SAC) gluconate formed by combining Sigma Anti-bonding Molecule Calcium Carbonate (SAC) with glucose, as an active ingredient.

2. The pharmaceutical composition according to claim 1, wherein the composition includes the SAC gluconate in an amount of 0.1 to 30% by weight (“wt. %”) based on a total weight of the composition.

3. The pharmaceutical composition according to claim 1, wherein the SAC has a constitutional composition of: 98 to 99 wt. % of calcium carbonate (CaCO₃); and
1 to 2 wt. % of any one or more selected from the group consisting of Na₂O, MgO, SiO₂, Fe₂O₃, K₂O, MnO₂, TiO₂, Al₂O₃, Cl, S, P, Cu and Ni.

4. The pharmaceutical composition according to claim 1, wherein the SAC is extracted from oyster shells and/or corals.

5. The pharmaceutical composition according to claim 1, wherein the composition increases a concentration of 17β-estradiol in blood.

6. The pharmaceutical composition according to claim 1, wherein the composition decreases concentrations of osteocalcin and CTx in blood.

7. The pharmaceutical composition according to claim 1, wherein the composition increases a bone mineral density (BMD), and concentrations of calcium and phosphorous.

8. The pharmaceutical composition according to claim 1, wherein the calcium-related degenerative disease is osteoporosis.

9. The pharmaceutical composition according to any one of claims 1 to 8, wherein the calcium-related degenerative disease is derived by ovariectomy or menopausal.

10. The pharmaceutical composition according to claim 1, wherein the cancer is one or more selected from colorectal cancer, rectal cancer, breast cancer, prostate cancer, pancreatic cancer, brain tumor, lung cancer, liver cancer, stomach cancer, bone cancer, lymphatic cancer, bone marrow cancer and hematologic malignancy.

11. A health functional food composition for preventing or improving calcium-related degenerative diseases, osteoporosis or cancers, comprising Sigma Anti-bonding Calcium (SAC) gluconate formed by combining Sigma Anti-bonding Molecule Calcium Carbonate (SAC) with glucose, as an active ingredient.

12. An animal feed composition for preventing or improving calcium-related degenerative diseases, osteoporosis or cancers, comprising Sigma Anti-bonding Calcium (SAC) gluconate formed by combining Sigma Anti-bonding Molecule Calcium Carbonate (SAC) with glucose, as an active ingredient.
